SEQUENCE LISTING (2 sequences)

SEQ ID NO: 1 - protein, 88 aa, Homo sapiens
MGSALRCGECGRCFTRPAHLIRHRMLHTGERPFPCTECEKRFTERSKLIDHYRTHTGVRPFTCTVCGKSFIRKDHLRKHQRNHAAGAK

SEQ ID NO: 2 - DNA, 264 bp, Homo sapiens
atgggctcagctttacgttgcggagaatgtggtcggtgctttacacgtccagcgcatctgattagacatagaatgctgcatactggtgaacgtccgttcccttgcactgagtgtgaaaaacgttttaccgagaggtctaaacttattgatcattatcgtacgcatacaggggttcggccgtttacgtgtaccgtttgtggcaaaagtttcatccgcaaggatcacttgcgaaaacatcagcgcaatcacgcagcaggtgccaaa

(19) United States
(12) Patent Application Publication
(10) Pub. No.: US 2025/0340606 A1
(43) Pub. Date: Nov. 6, 2025

(54) ZINC FINGER DOMAIN, AND METHOD FOR RECOVERING COBALT IONS BY USING SAME

(71) Applicant: INDUSTRIAL COOPERATION FOUNDATION JEONBUK NATIONAL UNIVERSITY, Jeonju-si, Jeollabuk-do (KR)

(72) Inventors: Seung Jae LEE, Jeonju-si, Jeollabuk-do (KR); Chung Woon YOON, Daegu (KR); Chae Min LEE, Jeonju-si, Jeollabuk-do (KR); Yun Ha HWANG, Jeonju-si, Jeollabuk-do (KR)

(21) Appl. No.: 18/728,581

(22) PCT Filed: May 26, 2022
(86) PCT No.: PCT/KR2022/007499

§ 371 Date: Jul. 12, 2024

(30) Foreign Application Priority Data
Jan. 12, 2022 (KR) 10-2022-0004732

(51) Int. Cl. C07K 14/47; C12N 15/70

(52) U.S. Cl. CPC C07K 14/47; C12N 15/70

(57) ABSTRACT

An embodiment provides a method for using a zinc finger protein in the selective recovery of metal ions included in various types of industrial waste, and provides: a PARIS zinc finger domain having an amino acid sequence of SEQ ID NO: 1; a composition for adsorbing and recovering cobalt ions, comprising the PARIS zinc finger domain as an active ingredient; and a method for recovering cobalt ions, comprising a step of culturing, in a material comprising waste cobalt, E. coli into which the PARIS zinc finger domain has been introduced.

### TECHNICAL FIELD

The present invention relates to a method of recovering metal ions, especially cobalt ions, using a zinc finger domain in the selective recovery of metal ions contained in various industrial wastes.

### BACKGROUND ART

The need for metal recovery technology is emerging due to the development of the 4th industry, such as the development of electric vehicles (batteries). Considering the toxicity and scarcity of metal ions, the ability of peptides to bind to specific metal ions may be an important means for environmental protection and metal ion recovery. In particular, cobalt is a rare earth metal accounting for only about 0.0025% of the Earth's crust, and is an essential material that is used in major industrial fields of the 4th industrial revolution, such as smartphones and electric vehicle batteries. Therefore, there continues to be a need for regeneration and recovery of cobalt.

One-third of proteins in biological systems activate their biological functions by binding to specific metal ions to perform their structural and functional roles in nature. Therefore, these proteins can be used to remove and recover rare metal ions from contaminated water or soil.

Zinc finger (ZF) proteins are transcription and translation regulators in eukaryotes and prokaryotes and are in vivo metalloproteins that form tertiary structures by coordination with metal ions. Zinc finger proteins have a distinct feature as metalloproteins, because the specific secondary folding in local zinc finger domains is generated in the presence of zinc ions.

Zinc finger domains are classified into classical and non-classical zinc finger domains depending on the ligands to which they bind. In the case of the classical zinc finger domain, Cys₂His₂ ligands bind to zinc ions, and in the case of the non-classical zinc finger domain, ligands such as Cys₂His₁Cys₁ bind to zinc ions. In general, cysteine (Cys) and histidine (His) bind to zinc ions to form the geometric structure of the zinc finger protein and activate the function of the protein.

In the case of existing studies on metal recycling using microorganisms, studies have been conducted focusing on either microbial fuel cells or microorganisms to which substances such as peptides are bound to the outside.

Korean Patent Application No. 10-2011-0042226 relates to a method of removing heavy metals or recovering precious metals using a microbial fuel cell. It discloses that a microbial fuel cell (MFC) can be used to remove Hg²⁺ in the form of a solid precipitate or deposit of metal Hg or Hg₂Cl₂ from wastewater containing heavy metals or precious metals, can incidentally produce power, and is capable of long-term economic operation without generating by-products.

Korean Patent Application No. 10-2011-0145668 relates to silver metal recovery and power production from wastewater using a microbial fuel cell. It discloses a method of recovering silver metal by a high-performance microbial fuel cell using silver ion wastewater as an electron acceptor and organic matter as an electron donor, which has the advantage of recovering silver metal while producing power in a cost-effective manner.

Korean Patent Application No. 10-2018-0004636 relates to a microbial adsorbent and a method of continuously recovering lithium using the same. It discloses that, when the microbial adsorbent comprising a support and a microorganism having a lithium-binding peptide displayed on the surface thereof is used for lithium recovery, continuous recovery of lithium ions from wastewater or seawater is possible.

In addition, there has been proposed a method capable of separating and recovering heavy metals, generated in the metal smelting process, using the principle that a silk protein in waste generated during silk processing reacts with metal ions to form a complex compound (Korea Silk Research Institute, 2015). According to this principle, when the complex compound is heated above a certain temperature based on the high melting points of silk protein and metal, it is possible to recover pure metal.

### DISCLOSURE

### Technical Problem

An object of the present invention is to propose a method of achieving the recovery of metal ions, which are finite resources, especially cobalt ions, by using the specific adsorption of metal ions by a zinc finger domain.

### Technical Solution

The present invention provides a PARIS zinc finger domain having the amino acid sequence of SEQ ID NO: 1, a gene encoding the same, a recombinant expression vector containing the gene, and a transformant obtained by introducing the recombinant expression vector into a host cell.

The present invention also provides a method for producing a PARIS zinc finger domain, comprising steps of: culturing the transformant; and isolating the PARIS zinc finger domain from the culture of the transformant.

The present invention also provides a composition for adsorbing and recovering cobalt ions, comprising the PARIS zinc finger domain as an active ingredient.

The present invention also provides a method for recovering cobalt ions, comprising a step of culturing E. coli, into which the PARIS zinc finger domain has been introduced, in a material containing waste cobalt.

### Advantageous Effects

According to the present invention, it is possible to use a zinc finger domain, which essentially requires binding with metal ions to maintain its structure and has high metal-binding specificity, as a tool for selective recovery of metal ions. In particular, it has been found that the protein adsorbs metal ions in the presence of cobalt ions. Thus, it is possible to achieve efficient recovery of cobalt ions using the zinc finger domain.

### BRIEF DESCRIPTION OF DRAWINGS

FIG. 1 shows the amino acid sequences of zinc finger domains in PARIS.

FIGS. 2A and 2B depict graphs showing the results of confirming specific adsorption of Co²⁺ ions by measuring the absorbance of apo-PARIS_ZF2-4 (a) and Co²⁺-PARIS_ZF2-4 (b).

FIG. 3 shows the results of measuring metal ions in E. coli following overexpression of PARIS_ZF2-4.

FIG. 4 shows the results of measuring the concentration of Zn ions in PARIS_ZF2-4 in cells when different concentrations of Co, Cu, and Fe ions were added to culture media.

FIGS. 5A, 5B and 5C show the results of measuring the concentrations of metal ions in PARIS_ZF2-4 in cells when different concentrations of Co, Cu, and Fe ions were added to culture media.

### BEST MODE

In the present invention, the absorption of metal ions in E. coli was confirmed using metals (Co, Cu, Fe) other than zinc that form coordination bonds with a zinc finger domain, and it was confirmed that cobalt (Co) formed the structure of the zinc finger domain instead of zinc and was involved in the zinc transport process of the protein. Accordingly, the present invention provides a method of using the zinc finger domain to selectively remove and recover metal ions.

In the present invention, in order to confirm specific adsorption of metal ions, a zinc finger domain was selectively expressed and purified. The present invention provides a method of specifically adsorbing metal ions using the zinc finger domain in E. coli. The metal ions may be metal ions other than zinc ions, specifically cobalt ions.

Specifically, the present invention provides a PARIS zinc finger domain having the amino acid sequence of SEQ ID NO: 1. In addition, the present invention provides a gene encoding the PARIS zinc finger domain, a recombinant expression vector containing the gene, and a transformant obtained by introducing the recombinant expression vector into a host cell.

The PARIS zinc finger domain consists of three zinc finger domains (PARIS_ZF2-4) present at the C-terminus of the parkin-interacting substrate (PARIS), and is a classical zinc finger domain.

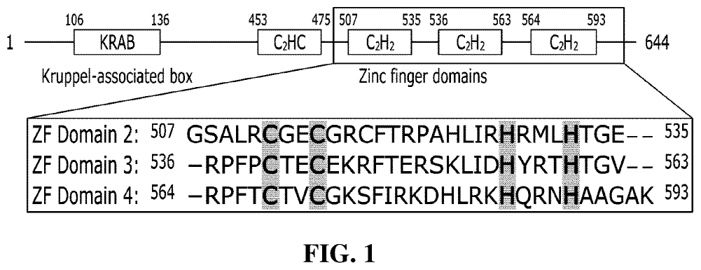

The PARIS regulates the hydrolysis of ubiquinone in the brain and consists of 644 amino acids. As shown in FIG. 1 and SEQ ID NO: 1, the PARIS has one non-classical Cys₂His₁Cys₁ zinc finger domain and three classical Cys₂His₂ zinc finger domains. In the present invention, the Cys₂His₂ domains, that is, the three classical zinc finger domains, are applied to E. coli to confirm their binding to metal ions. The three zinc finger domains are sequentially located from the second domain among a total of four zinc finger domains of PARIS, and are thus called PARIS_ZF2-4 (see FIG. 1).

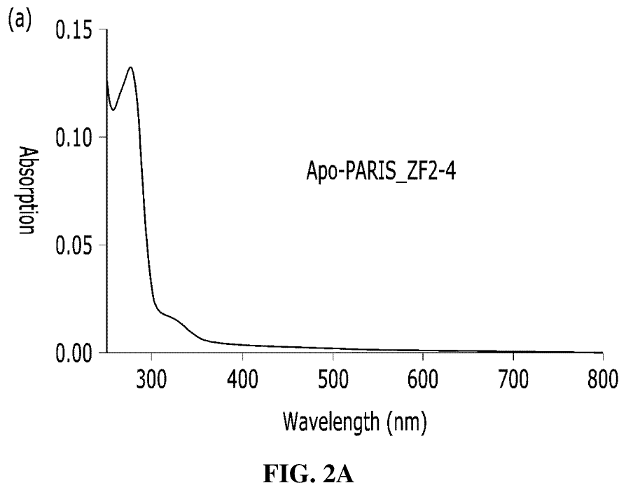

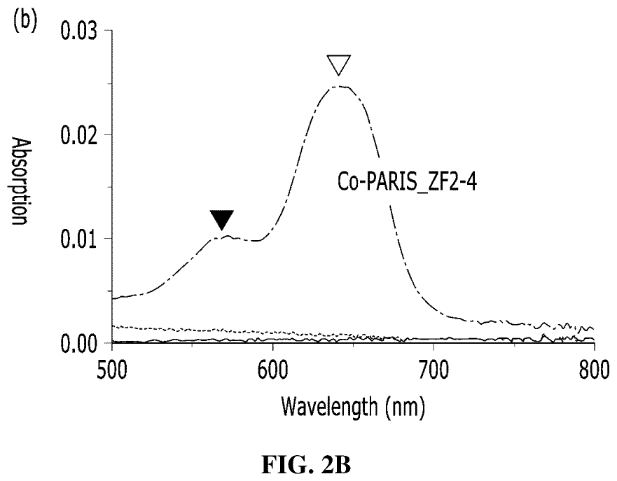

The PARIS_ZF2-4 can basically form coordination bonds with metal ions. Thus, zinc ions in PARIS_ZF2-4 may be replaced by different metal ions, wherein the different metal ions may be metal ions of Co²⁺, Cu²⁺, and Fe³⁺. Replacement with the different metal ions can be confirmed through the peaks of ligand-to-metal charge-transfer (LMCT) or d-d transition bands that specifically occur upon metal replacement, using a UV/Vis spectrophotometer (see FIGS. 2A and 2B).

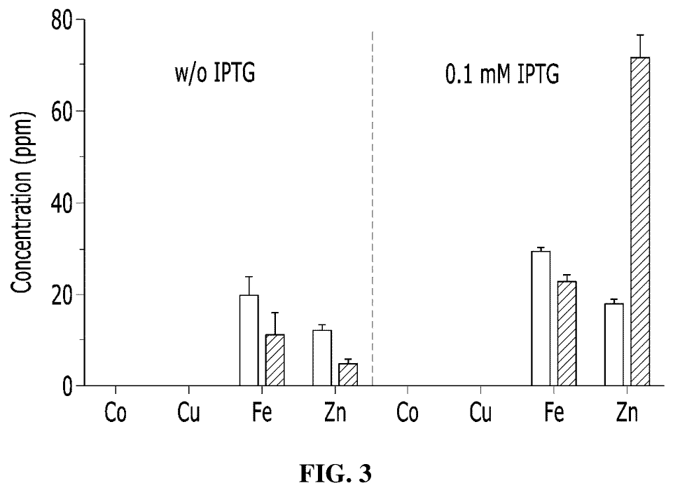

It was confirmed that the PARIS_ZF2-4 was successfully expressed in E. coli and bound to zinc ions. Specifically, it was confirmed that the concentration of zinc ions in the cells increased when the protein was expressed (see FIG. 3).

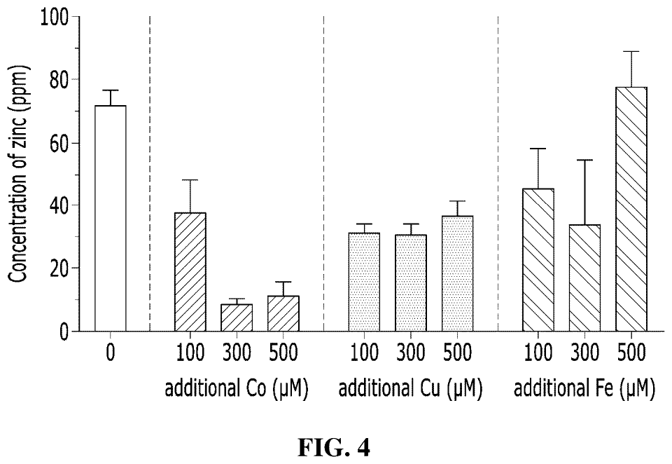

E. coli containing the PARIS_ZF2-4 was cultured under culture medium conditions with different concentrations of different metal ions, and the concentration of zinc ions in the cells was measured using ICP-OES (inductively coupled plasma-optical emission spectrometry). As a result, it was confirmed that the concentration of zinc ions in the cells specifically decreased when the protein was expressed in the presence of covalent metal ion (Co²⁺) among the above-described different metal ions (see FIG. 4).

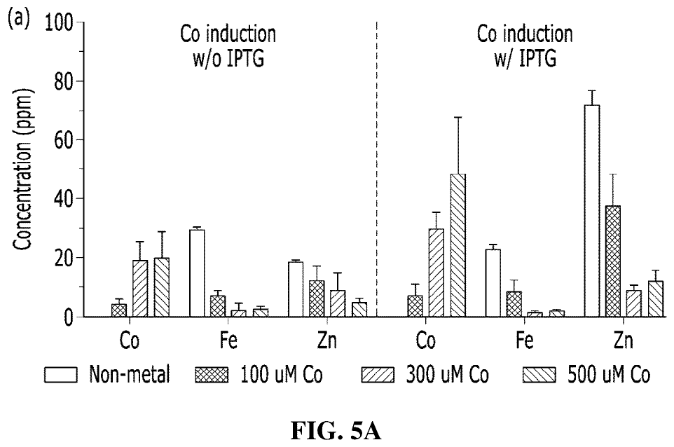

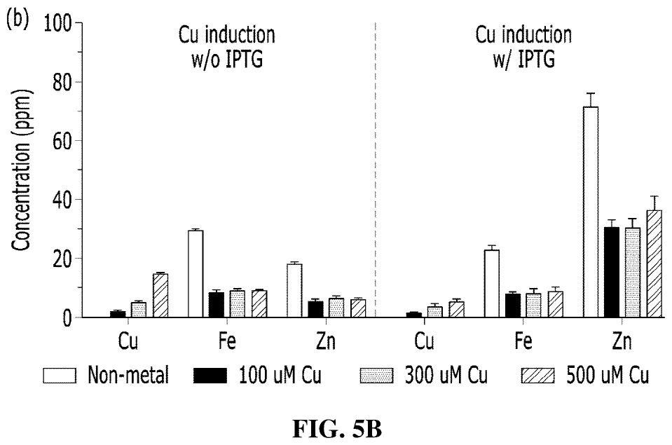

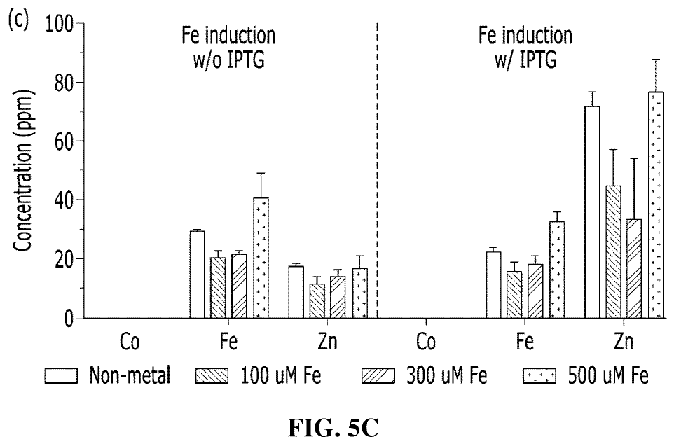

Additionally, E. coli containing PARIS_ZF2-4 was cultured under culture medium conditions with different concentrations of different metal ions, and the concentrations of the different metal ions in the cells were measured using ICP-OES (inductively coupled plasma-optical emission spectrometry). As a result, it was confirmed that, as the concentrations of covalent metal ions (Co²⁺) among the different metal ions increased, the concentration of zinc ions in the cells specifically decreased, while the concentration of cobalt ions in the E. coli increased. That is, the coordination bond with the zinc ion in the E. coli was replaced by a coordination bond with the cobalt ion (see FIGS. 5A, 5B and 5C).

Therefore, the present invention provides a method for producing a PARIS zinc finger domain, comprising steps of: culturing a transformant containing the PARIS zinc finger domain; and isolating the PARIS zinc finger domain from the culture of the transformant.

The present invention also provides a composition for adsorbing and recovering cobalt ions, comprising the PARIS zinc finger domain as an active ingredient. The zinc finger domain may be a classical or non-classical zinc finger domain. Preferably, the PARIS zinc finger domain is a classical zinc finger domain. Additionally, preferably, the PARIS zinc finger domain may be free of zinc and may be apo-PARIS_ZF2-4 obtained by removing Zn²⁺ from PARIS_ZF2-4.

The present invention also provides a method for recovering cobalt ions, comprising a step of culturing E. coli, into which the PARIS zinc finger domain has been introduced, in a material containing waste cobalt. Preferably, cobalt ions are present at a concentration of 100 μM or more in the material containing waste cobalt. The PARIS zinc finger domain introduced into E. coli can achieve recovery of cobalt ions during or after its expression in E. coli.

Hereinafter, the present invention will be described in detail by way of examples.

However, the following examples are intended to illustrate the present invention in detail, and the content of the present invention is not limited by the following examples.

### MODE FOR INVENTION

### Example 1-1: Cloning and Transformation of PARIS_ZF2-4-pET15b

In order to express and purify the zinc finger domain having the amino acid sequence of SEQ ID NO: 1, a recombinant expression vector was constructed into which a PCR product comprising the nucleotide sequence of SEQ ID NO: 2 has been inserted. As a result of performing nucleotide sequencing on the recombinant expression vector constructed as described above, it was confirmed that the nucleotide sequence of SEQ ID NO. 2 was properly inserted. The recombinant expression vector was transformed into E. coli.

The transformant obtained as above was streaked on 10 mL of LA solid medium (LB medium+50 μg/mL ampicillin) and cultured at 37° C. for 12 hours or more. A single colony appearing on the solid medium was inoculated into a test tube containing 5 mL of LB medium and 50 μg/mL ampicillin through a single colony isolation process, and was seed-cultured in a shaking incubator at 37° C. for 12 hours or more. The transformant obtained as above was freeze-cultured in 15% glycerin solution before use.

### Example 1-2: Overexpression and Purification of PARIS ZF2-4-pET15b

The freeze-stored transformant in Example 1-1 was inoculated into a test tube containing 200 mL of LB medium and 50 μg/mL ampicillin, and was seed-cultured in a shaking incubator at 37° C. for 12 hours or more. Thereafter, 5 mL of the seed culture was added to a 2,000 mL flask containing 500 mL of LK medium and subjected to a total of 2 L of main culture. When the absorbance at 600 nm reached 0.6, isopropyl-1-thio-β-D-galactopyranoside (IPTG) was added to a final concentration of 0.5 mM to induce overexpression of the protein having the amino acid sequence of SEQ ID NO: 1. In the process of inducing overexpression of the protein having the amino acid sequence of SEQ ID NO: 1 as described above, the stirring speed was adjusted to 200 rpm and the culture temperature was maintained at 37° C. After addition of IPTG, the stirring speed was adjusted to 200 rpm, the culture temperature was adjusted to 25° C., and culture was performed for 6 hours.

In addition, the culture of the transformant in which overexpression of the protein having the amino acid sequence of SEQ ID NO: 1 has been induced as described above was dispensed into a 500 mL PP tube and centrifuged at 8,000 rpm at 4° C. for 20 minutes, and the supernatant was removed. 50 mL of buffer A (25 mM MOPS, 50 mM NaCl, 5 mM MgCl₂, 0.01 μL/mL DNaseI, 0.002 mg/mL, pH 6.5) was added to the pellet which was then lysed by sonication to obtain a cell lysate of the transformant.

The cell lysate obtained as described above was centrifuged at 12,000 rpm for 1 hour at 4° C., and the supernatant was collected. The overexpressed protein having the amino acid sequence of SEQ ID NO: 1 was isolated from the collected supernatant using fast protein liquid chromatography equipped with an SP-Sepharose ion exchange column and Superdex 75.

### Example 2: Study on Metal Ion Adsorption of PARIS ZF2-4

To obtain apo-PARIS_ZF2-4 protein from which metal ions were excluded, the protein isolated and purified in Example 1-2 was acid-treated in 1 M HCl at 200 rpm at 4° C. for 1 hour. For metal ion adsorption, 3 equivalents of cobalt (II) chloride hexahydrate (Sigma-Aldrich)) was added, and the absorbance of the solution was measured using a UV/Vis spectrophotometer. The results are shown in FIGS. 2A and 2B. The characteristic d-d transition bands (560 nm and 644 nm) indicating the adsorption of cobalt ions by the zinc finger domain were confirmed, and the UV/Vis spectral results of apo-PARIS_ZF2-4 were used as a control.

### Example 3: Measurement of Concentration of Metal Ions in E. coli

In order to confirm the coordination bond with zinc ions, which is formed upon expression of the zinc finger domain, the concentrations of metal ions before (w/o IPTG) and after (0.1 mM IPTG) expression of the zinc finger domain in E. coli were examined. The cultured cells were separated into the cell culture medium (LB medium) and the cells, and the concentrations of metal ions contained in the cell culture medium and the concentration of metal ion in the E. coli cells were measured using ICP-OES (ThermoFisher Scientific, iCAP 7000). The results are shown in FIG. 3. The white bar represents the concentration of metal ions in the supernatant before cell lysis, and the hatched bar represents the concentration of metal ions in the supernatant after cell lysis.

Here, the concentration of zinc metal ions in E. coli in which the expression of PARIS_ZF2-4 was induced by IPTG increased compared to the concentration of metal ions in the cell culture medium. Specifically, it was confirmed that the three zinc finger domains in PARIS_ZF2-4 formed coordination bonds with zinc ions when expressed in E. coli.

### Example 4: Expression of PARIS ZF2-4 in Presence of Co, Cu and Fe

During the expression of PARIS_ZF2-4 in E. coli, different metal ions were added at different concentrations, and changes in the concentrations of zinc ions in the E. coli were measured. The results are shown in FIG. 4. As shown therein, when the concentrations of zinc ions in the presence of the different metal ions, specifically additional metal ions of cobalt, copper and iron, were measured using ICP-OES (iCAP 7000, ThermoFisher Scientific), it was confirmed that the concentrations of zinc ions was reduced by the addition of the metal ions. It was confirmed that the concentration of zinc ions specifically decreased in the presence of one type of metal ions (cobalt ions) among the different metal ions, indicating that cobalt ions formed coordination bonds with zinc ions by replacing zinc ions in E. coli expressing PARIS_ZF2-4.

### Example 5: Evaluation of Metal Ion Recovery Ability after Expression of PARIS ZF2-4

In order to evaluate the ability of PARIS_ZF2-4 to recover different metal ions, the ions were added in the concentration range of 100 to 500 M, and the concentrations of metal ions in the E. coli were measured using ICP-OES (iCAP 7000, ThermoFisher Scientific). Replacement of the zinc metal ions coordinated to PARIS_ZF2-4 was evaluated by comparing the concentrations of the added metal ions and the reduced zinc ions in the E. coli. The results are shown in FIGS. 5A, 5B and 5C.

Here, it was confirmed that, when among the different metal ions, one type of metal ions, specifically cobalt ions (100, 300, and 500 μM), were added, the concentration of cobalt ions in E. coli expressing PARIS_ZF2-4 increased and the concentration of zinc ions in E. coli expressing PARIS_ZF2-4 decreased. In addition, it was confirmed that, as the concentration of cobalt ions increased, the amount of cobalt ions recovered by PARIS_ZF2-4 in the E. coli increased. Specifically, it was confirmed that the zinc ions forming coordination bonds with PARIS_ZF2-4 in the E. coli were replaced by cobalt ions in the presence of a high concentration of cobalt ions.

### INDUSTRIAL APPLICABILITY

The present invention relates to a method of recovering metal ions, especially cobalt ions, using a zinc finger domain in the selective recovery of metal ions contained in various industrial wastes. According to the present invention, it is possible to use a zinc finger domain, which essentially requires with metal ions to maintain its structure and has high metal-binding specificity, as a tool for selective recovery of metal ions. In particular, it has been found that the protein adsorbs metal ions in the presence of cobalt ions. Thus, it is possible to achieve efficient recovery of cobalt ions using the zinc finger domain.

1. A PARIS zinc finger domain having the amino acid sequence of SEQ ID NO: 1.

2. A gene encoding the PARIS zinc finger domain of claim 1.

3. A recombinant expression vector containing the gene of claim 2.

4. A transformant obtained by introducing the recombinant expression vector of claim 3 into a host cell.

5. A method for producing a PARIS zinc finger domain, comprising steps of:
culturing the transformant of claim 4; and
isolating a PARIS zinc finger domain from the culture of the transformant.

6. A composition for adsorbing and recovering cobalt ions, comprising the PARIS zinc finger domain of claim 1 as an active ingredient.

7. The composition of claim 6, wherein the PARIS zinc finger domain is a classical or non-classical zinc finger domain.

8. The composition of claim 6, wherein the PARIS zinc finger domain is free of zinc and is apo-PARIS_ZF2-4 obtained by removing Zn²⁺ from PARIS_ZF2-4.

9. A method for recovering cobalt ions, comprising a step of culturing E. coli, into which the PARIS zinc finger domain of claim 1 has been introduced, in a material containing waste cobalt.

10. The method of claim 9, wherein cobalt ions are present at a concentration of 100 μM or more in the material containing waste cobalt.

11. The method of claim 9, wherein the PARIS zinc finger domain introduced into E. coli is or has been expressed in E. coli.
